(19) United States
(12) Patent Application Publication
(10) Pub. No.: US 2002/0054892 A1
(43) Pub. Date: May 9, 2002

(54) Ultrasonic control of crystallisation

(75) Inventors: Xavier Garijo, Trumbull, CT (US); Renoo Avinash Blindt, Bedford (GB); Maria Patrick, Bedford (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/902,292

(22) Filed: Jul. 10, 2001

(30) Foreign Application Priority Data
Jul. 18, 2000 (GB) 0017626.3
Jul. 18, 2000 (GB) 0017623.0

(51) Int. Cl. A61K 7/00

(52) U.S. Cl. 424/401; 204/157.62

(57) ABSTRACT

A method of inducing solidification in a fluid composition comprising a cosmetic active, a crystalline organic structurant and a carrier, comprising exposing the fluid composition to ultrasound or converting the composition to a soft solid.

A cosmetic stick in the form of a soft solid or firm stick comprising a cosmetic active, a crystalline organic structurant and a carrier in which the organic structurant is present in domains that are deficient in the cosmetic active, such as in particular an antiperspirant or deodorant active.

The present invention relates generally to a method of modifying the hardness of fluid composition which has cooled to a solid, and also to modifying the solidification temperature of the fluid as it is about to solidify. It is particularly useful for inducing solidification in fluids. More specifically, the present invention relates to a method of inducing solidification in a cooling fluid, and altering by increasing or reducing the hardness of the solidified fluid by exposing the fluid to ultrasound during solidification. In a preferred aspect, the invention has utility in the manufacture of cosmetic products, such as sticks, soft solids and creams.

Typically, a solid or semisolid product such as a cosmetic stick product may be made by solidifying, a fluid formulation of that product, which may be a molten liquid, solution, suspension or emulsion, conveniently by cooling, ie introducing structure into the fluid phase. Many products (such as but not necessarily limited to cosmetic sticks and soft solids containing a cosmetic active) may be made by heating a number of components of the end product together to produce a fluid molten solution, suspension or emulsion of the cosmetic active, since such heating and mixing is a convenient way of intimately mixing and/or otherwise combining the components of the product. The molten fluid may then be formed into the topical product, for example by dosing into a product dispensing container, before cooling. The typical way of forming such products from molten solutions, suspensions or emulsions is by melt casting them, though if the resulting product is intended to be a soft solid or cream, the cooling of the product may typically be accompanied by shearing the product.

When products are made from molten fluids in this way, the product is then invariably subjected to a cooling process once it is in its dispensing container. This may be for a variety of reasons. When the product has been melt cast from a molten fluid, cooling may facilitate the manufacturing and/or packaging process for that product, since it may not be possible or desirable to have molten formed products in a manufacturing environment for longer than is necessary. This may be because of the difficulty of transporting a molten product, which may otherwise “slop” around, or because of deterioration of the final product which could otherwise occur if it is kept at too high a temperature for too long without cooling.

The problem may be particularly acute where the molten product is in the form of a suspension, since if the molten product is left for too long, the suspended solid phase can settle under the effect of gravity, causing a non-uniform distribution of the suspended phase in the final solid or semi-solid product. Hence, active cooling of products made from molten fluids is usually desirable, especially when the molten product contains a suspended solid phase.

The rate of cooling, and how far the fluid has been heated above its normal solidification point both determine how quickly the fluid solidifies to attain a viscosity sufficient to ameliorate the various processing problems outlined hereinabove.

It is common for a process for the manufacture of a such products to include a step in which a liquid or molten product must be solidified, and typically this is achieved by passing the product shortly after having being formed (e.g. by melt casting into a dispensing container) through a cooling tunnel. On an industrial scale, this stage of the process is often relatively slow, and as a result may significantly increase the overall processing time. Increased processing times are obviously undesirable, because they decrease the throughput of the process and increase the cost of manufacturing the product.

Cooling tunnels are also generally very expensive to run, and there is typically the need to use refrigeration coolants to produce cool air to cool the product. The provision of a cooling tunnel also increases the cost of manufacture of the product due to the necessity for additional capital investment in a refrigeration plant, and additional operating expenditure. As such, cooling tunnels are not parts of the production line which can be readily proliferated without high capital cost. The cooling tunnel part of the production line also often represents a bottleneck in the process, because of the time that a molten product necessarily needs to spend in the tunnel.

In the art of metallurgy, it is known that exposure of cooling molten metal system, (which are typically aluminium or copper based systems) to ultrasound can cause an acceleration in crystallisation rate, which can have the effect of elevating the solidification temperature of a cooling molten solution, and also of modifying crystal structure in the resultant cooled metal matrix.

U.S. Pat. No. 5,209,879 describes the use of forces such as ultrasound to modify the crystallized form of certain naturally occurring triglycerides from the α form to the β form for use in manufacture of encapsulates. Likewise, EP-A-765,605 discloses the use of ultra-sonic energy to accelerate the transformation of unstable and semi-stable polymorphic crystals in edible fat compositions. Such teachings cannot be generalised to most organic structurants for cosmetic carriers which do not display different crystalline forms at ambient to mildly elevated temperatures and accordingly does not offer direct teaching for processes to produce a stick cosmetic.

U.S. Pat. No. 5,471,001 describes the crystallisation of adipic acid from aqueous solution whilst being irradiated with low intensity ultrasound, in order to produce crystals with smooth surfaces. This does not teach how to structure a water-immiscible carrier in a stick cosmetic.

U.S. Pat. No. 5,830,418 discloses the use of ultrasound to promote crystallisation of solid substances in a flowable material on a take-up belt or drum. This offers no teaching relating to the formation of a structured carrier.

WO 92/20420 (EP-B-584127) relates to a method and apparatus for solidifying a liquid by subjecting the liquid to a physical disturbance to induce vibration and/or cavitation in the liquid so as to control the nucleation and/or crystal growth steps of the solidification process. The text contemplates subjecting frozen material, eg ice crystals to ultrasound and exemplifies lipid phase changes in water-in oil emulsions such as cream. It also discloses the precipitation of fatty acids from ultrasound-exposed and cooled samples of olive oil. However, the text does not teach the structuring of a water-immiscible cosmetic carrier.

GB-A-2,089,230 describes a process for purifying various materials, including acetic acid, by crystallising them from solution whilst subjecting them to low frequency ultrasonic waves. Once again, this provides no teaching in regard to forming a structured water-immiscible carrier for a cosmetic stick.

WO98/23350 and WO 99/59709 both disclose a process for preparing a dispersion of droplets of a melted organic compound having a ΔH/RT value of above 10 in an aqueous phase optionally in the presence of an emulsifier in which the composition is subjected to ultrasonic vibration whilst it is being cooled and vigorously agitated to accelerate crystallisation of the organic compound. However, the text offers no guidance to the formation of a structured water-immiscible carrier for a cosmetic formulation.

Typical cosmetic products such as cosmetic sticks often have a structure which reflects the cooling regime to which they have been subjected. Thus, in a typical antiperspirant stick which has been made by melt casting and then cooled in a cooling tunnel, if a lateral cross section of the stick is viewed, it is often possible to make out an outer region of stick adjacent the stick dispensing container, which is typically about 2 mm thick, which is a so-called “chill zone” of relatively short random orientation crystals. At the core of the stick, adjacent the screw thread (if there is one), there is usually found a so-called equiaxial zone of comparatively long (i.e. longer than those in the chill zone), relatively random orientation crystals.

In between these two zones, there is a middle area of crystals in what may be known as the dendritic zone which are relatively long (i.e. typically longer than those in either the chill zone or the equiaxial zone), and are axially orientated. The crystals tend to comprise the organic structurant, such as the waxes and organic structurants which have crystallized during the cooling of the molten stick, such as for example stearyl alcohol. In sticks which are based on wax or other organic crystalline structurants, it is this crystal structure which is thought to contribute significantly to the physical and sensory properties of the stick, including in particular the feel of the stick on application. However, a contribution to the sensory properties of the stick may also be attributable to the particle size of any particulate antiperspirant active in the composition, and how it is positioned or embedded in the crystal structure.

We have surprisingly found that it is possible to accelerate the rate of crystallisation in organic structurant based systems when they are cooled, and also to modify the resultant crystal structure of solidifying non-metallic systems, by exposure of the cooling system to ultrasound. The invention has utility in particular in the formation of crystalline organic matrices, such as are typically found in cosmetic stick products.

In addition, when the stick composition contains suspended cosmetic active solid, such as a suspended antiperspirant active, it has been found that the suspended solid is typically less embedded in the crystalline structurant network, in contrast to many prior art melt cast sticks containing suspended solids.

In a further embodiment, we have that it is possible by using the invention to produce using traditional melt casting methods sticks which have a different crystal structure to those conventionally produced, where melt casting is followed by cooling in a cooling tunnel, with a consequent modification of stick properties.

Thus, according to a first aspect of the invention, there is provided a method of inducing solidification in a fluid composition comprising a cosmetic active, a water-immiscible carrier and an organic crystalline structurant, comprising exposing the fluid composition to ultrasound.

In more detail, there is provided a method of manufacturing a cosmetic stick comprising providing a molten composition of the cosmetic stick containing a cosmetic active, a water-immiscible carrier and an organic structurant which is crystalline when the composition is solid, the molten composition optionally containing suspended solid particles, cooling the molten composition to a temperature at which it is solid, and subjecting the molten composition to ultrasound prior to solidification of the composition.

According to a further aspect of the invention, there are provided cosmetic sticks particularly cosmetic sticks containing a suspended active component, made according to the method of the invention.

Sticks herein can comprise either firm sticks or soft solids.

Exposure to ultrasound may be carried out prior to the cooling fluid reaching its solidification temperature and cease before the solidification temperature is reached, or it may be continued whilst the cooling fluid passes through its solidification temperature.

In a typical utility, the method will have the effect of causing a fluid molten composition comprising an organic crystalline structurant such as (but not limited to) a wax, which is at least partially liquid in the fluid or molten composition but which is normally at least partially solid at room temperature (e.g. 23° C.), when cooling to solidify at a temperature which is above its normal solidification temperature. This may have the overall effect of increasing the solidification temperature of the cosmetic stick, compared to what it would have been for the same composition of stick which is not exposed to ultrasound during its manufacture, during for example melt casting, ie the onset of solidification occurs in a cooling process at a higher temperature when the melt is exposed to ultrasound compared with when it is not so exposed. However, the method may also be used to speed up the rate of crystallisation of a molten fluid at or very close to its normal solidification temperature.

Exposing the cooling fluid to ultrasound may also have the effect of modifying the crystal structure compared to the typical structure of a melt cast stick described above.

Thus, according to an aspect of the present invention, there is provided a cosmetic stick comprising a cosmetic active in a carrier that contains a crystalline organic structurant which is characterised by the structurant forming domains that are deficient in the cosmetic active.

Domains herein that are deficient in the cosmetic active, such as the antiperspirant active are discrete crystalline regions comprising essentially structurant. The significant deficiency of the cosmetic active can be determined by spectroscopic techniques that measure the concentration of a reference element within the domain such as conventional cryo-scanning microscopy techniques. Alternative spectroscopic techniques that the skilled man considers suitable for quantitative determination of an element in a region of a solid product may also be used. A reference element is one which indicates the presence of the cosmetic active and is absent from the structurant. By way of example, in the case of aluminium or zirconium-containing antiperspirant actives, both aluminium and zirconium are suitable elements to act as reference elements, because they are absent from the crystalline structurants. Such domains that are produced herein by exposure of the stick material to ultrasound are randomly oriented within the stick.

The method may also be used to initiate a wider range of solidification temperatures for a given cosmetic stick composition, thereby increasing the flexibility which can be obtained in a manufacturing environment.

Throughout this description, the phrase “solidification temperature” can also be used in relation to a composition which in fact exhibits a range for its solidification temperature. Where this is the case, the phrase “solidification temperature” will typically relate to the upper end of the solidification range, unless the context otherwise dictates. Also, by normal solidification temperature is meant the temperature at which a composition would solidify (or, in line with the above, start to solidify) when being cooled from a molten composition at atmospheric pressure which was not subject to any outside influence such as ultrasound.

A typical product of the process is a cosmetic stick, which term is used herein to include not only firm stick compositions which are rigid at room temperature, but also compositions which are soft solids and creams at room temperature.

The method has particular utility in the solidification of cooling molten—fluids containing an organic crystalline structurant, which may be liquids, solutions, suspensions or emulsions, to produce products which are sticks, soft solids or creams, though it is thought that the method is particularly suitable for use in the solidification of suspensions in molten solutions of crystalline organic structurant fluids. Where the method is used in the cooling of molten fluids, the use of the method can result in the solidification temperature of the crystalline organic structurant containing fluid composition being higher than it would otherwise be. The method is particularly suitable for manufacturing methods which utilize a cooling step (such as in a cooling tunnel) after casting a product from molten ingredients.

Organic crystalline structurant containing fluids which are suitable for use in methods according to the invention are any organic fluids which are capable or providing a crystalline structure in a manufactured product, thereby implying that crystalline organic structurant undergoes a degree of solidification during the cooling step. However, the method has particular utility where crystalline organic structurant is based on compounds containing high proportions of relatively long hydrocarbon chains. Particularly suitable crystalline organic structurants are waxes, which are as least partially molten in the fluid composition. In particular, the solidified crystalline organic structurant may cause the resultant product to be solid or semi-solid at room temperature.

Wax based compositions according to the invention have a structure which does not form fibres on solidification. Any such fibres in compositions which are outside the scope of the invention may readily be readily detected by known techniques, such as x-ray diffraction and optical microscopy. Crystalline wax compositions according to the invention may typically have a structure which is based on needle, plate or spherulite crystalline forms. Suitable non-fibre forming waxes include paraffin waxes, Synchrowax™ HRC, carnabau, beeswax, modified beeswaxes, microcrystalline waxes, polyethylene waxes and fatty ester derivatives of polyols, such as glycerol monostearate and related compounds. Other waxes include spermacetti, candelilla and silicone waxes, as well as fatty alcohols, fatty acids, esters containing a fatty acid or alcohol residue and waxy hydrocarbons. The fatty residue in alcohols, acids or esters commonly is linear and usually comprises at least 12 carbons, such as up to 30 and particularly from C16 to C22.

Also suitable for use according to the invention are hydrogenated unsaturated or polyunsaturated vegetable oils such as hydrogenated castor oil sometimes called castor wax.

The method has been found to have particular utility in the provision of cosmetic sticks, particularly wax and other long chain structurant material (e.g. fatty acid, fatty alcohol, and long chain fatty ester) based sticks. The method has also been found to be particularly useful in the manufacture of stick products for underarm use, such as deodorant sticks and particularly antiperspirant sticks, and -especially those sticks which contain suspended antiperspirant active salts.

Fluid compositions for which the invention has particular utility will conveniently have a normal solidification temperature in the region of 25-150° C., more conveniently in the region of 30-90° C. Conveniently fluid compositions when treated according to the invention will have their solidification temperatures raised by at least 1° C. or more, and preferably at least 3° C. or more.

The method and compositions according to the invention have the advantage that, at least in the context of cosmetic sticks which are melt cast, the stick can be caused to start to solidify at a higher temperature than it would otherwise do. In terms of a typical process in which the sticks may pass through a cooling tunnel as part of the cooling process after being cast, this means that for a given composition the residence time of the stick in the cooling tunnel may be less, or even in some circumstances a cooling tunnel may not be needed at all, since the temperature at which the stick solidifies is higher. It is no longer necessary to cool the stick to as low a temperature as was previously necessary to achieve the onset of solidification of the stick, and thereby avoid the disadvantages described above in relation to the importance of cooling.

A particular problem which may be ameliorated is undesirable and premature settling of any suspended solids where the cosmetic stick contains these, though as will be readily appreciated, a reduced residence period in a cooling tunnel will also reduce processing time and costs.

In addition to the processing advantages achievable by the invention as described above, other advantages are also obtainable. A conventional antiperspirant stick composition containing suspended active will typically have a non-uniform structure, as describe above. A typical antiperspirant stick which has been melt cast (which is the standard manufacturing method in the trade) comprises an equiaxial area, a dendritic zone and small crystals adjacent the barrel. The dendritic zone crystals are relatively long and radiate axially from the core area. The small crystals form on the outside of the stick and form a chill zone. This non-uniform crystal structure of the stick affects its sensory and mechanical properties.

It has been found that sticks made according to the method of the invention typically have a macro crystal structure which is more random in orientation, with the crystal sizes generally being smaller, typically a factor of ten smaller that those produced using traditional melt cast methods without using ultrasound in their processing. In other words, at the micro-structure level, the structurant is present in the form of domains. In some circumstances, this can provide different sensory attributes to the stick.

In addition to the above crystal structure of the stick matrix, in practice it is thought that sticks which are solidified by traditional cooling undergo a process by which solidification is actually initiated by nucleation. This can (though need not necessarily) be initiated by the presence of solid particles of suspended antiperspirant active. These act as “seeds”, causing the molten matrix material in their vicinity to solidify, and grow outwards. Crystallisation of the product matrix around these seeds may cause proliferation of the matrix itself. This can explain the presence of a significant level of for example aluminium or zirconium relative to carbon in cryoscanning electron microscopy in sticks solidified by a traditional cooling process.

A disadvantage of products which comprise a suspended solid active such as a suspended antiperspirant active in a hydrophobic matrix (e.g. one containing notable levels of materials such as waxes) is that the individual antiperspirant active particles may become effectively at least partially (and often fairly substantially) enrobed in the hydrophobic matrix materials. This coating may hamper the effectiveness of the antiperspirant active in use, and hence the topical composition, since in practice sweat is prevented from as readily or effectively combining with the active as it would if the active was not at least partially enrobed.

A surprising advantage we have found is that cosmetic sticks produced according to the invention with the same crystalline structurant and carrier have a macrocrystal structure different to those of the same empirical formulation produced according to conventional melt casting and cooling techniques. In particular, the structure has been found to be random orientation within the stick, and not to have a region of axially orientated dendritic zone structure, which is common to conventionally produced sticks. Such sticks may also have less of a coating of matrix hydrophobic material around any suspended active, and hence may have a higher efficacy. Sticks made in accordance with the present invention concentrate the structurant, and particular the wax structurant, in the aforementioned domains that are deficient in the cosmetic active.

The instant invention accordingly represents an especially suitable way of employing a wax structurant in the formation of cosmetic sticks, such as antiperspirant or deodorant sticks.

Hence, according to a further aspect of the invention, there is provided a cosmetic stick composition such as an antiperspirant for topical application to the human skin comprising a suspended active such as an antiperspirant active and a hydrophobic matrix, characterised in that the stick has a random non-fibrous crystalline structure throughout the body of the stick. The random crystal structure is particularly noticeable between the equiaxial zone and the chill zone areas of the stick.

According to a further aspect of the invention, there is provided a cosmetic stick composition such as an antiperspirant for topical application to the human skin comprising a suspended active such as an antiperspirant active and a hydrophobic matrix, characterised in that when the stick is analysed by cryo scanning microscopy techniques, the ratio of the size of the aluminium peak to carbon peak in the scanning micrograph is higher than when the same composition of stick is made by melt casting techniques.

According to a further aspect of the invention, there is provided a cosmetic stick, and particularly an antiperspirant stick composition for topical application to the human skin, made according to the process of the invention.

Aside from the processing advantages, stick compositions produced according to the invention have been found potentially to have a number of advantages over sticks produced by the traditional melt cast and cooling process. These include different sensory properties, including potential for reduced greasiness, reduced whitening on the skin on application, and may in some circumstances also have enhanced efficacy.

Such sticks of the present invention may also have other advantages, such as the possibility of controlling hardness, for example increased hardness but without any detrimental increase in brittleness or decreasing hardness, so as to offer an additional processing tool to produce a desired hardness or a range of different hardnesses from the same empirical formulation, the ability to cast and solidify the sticks at higher temperatures, and in the case of suspension sticks, more uniform distribution of suspended active, ie reducing sedimentation. This last benefit can lead to reduced irritation in suspension antiperspirant sticks, since a concentration of suspended active in one part of the stick can lead to irritation.

In relation to hardness, it has been found that for a given level and type of crystalline organic structurant, a stick made according to the invention may be made to have a higher or lower hardness than one made using conventional melt casting and cooling techniques. Hence, given the same composition, it is possible to make the stick generally harder or lower utilizing the method of the invention. Conversely, to attain a given stick hardness, a stick of the same hardness can be manufactured using less structuring materials using the process of the invention, compared to one made by conventional melt casting and cooling.

The difference in processing parameters produced by the use of ultrasound during cooling also provides the opportunity to formulate products with in particular different (e.g. softer), or lesser amounts of structurant materials, further providing the opportunity to produce cosmetic sticks with different and improved sensory properties and performance, as well as reduced costs, as described above.

The method of exposing the organic liquid to ultrasound may be any one conventional in the art. Techniques and equipment include the use of ultrasonic probes or transducers, which techniques will be familiar to those skilled in the art. If the composition ingredients are mixed in a batch fashion in a vessel, ultrasound can be introduced into the vessel. However, it is preferred that ultrasound be introduced into the composition by introducing it into a dosing pipe used to convey the molten product material into the individual dispensing container barrels.

In utilising the method of the invention, it has been found that the actual application of ultrasound to the composition may be attained in a variety of ways, and may suffer from relatively few constraints. It has also been found that when applying the ultrasound under set conditions, a stick formulation can demonstrate an increase in hardness, depending on the time and intensity of the ultrasound exposure, before peaking. As the stick formulation is then subjected to prolonged ultrasound exposure, the composition can then typically start to reduce in hardness before eventually attaining the same hardness that the composition would attain if not exposed to ultrasound. However, the ability to modify hardness is only one of the benefits that may be derived from using the method of the invention, as is outlined above.

A preferred method when casting antiperspirant sticks from a vessel of molten material of introducing ultrasound is to employ two ultrasound transducer rigs on the exterior of the vessel in which the ingredients are melted. The ideal frequency and intensity of ultrasound employed will be vessel and formulation dependent, but can readily be determined by the skilled man. The ideal frequency and intensity employed are such that the ultrasound waves become resonant in the vessel. The use of concentric ultrasound rigs described above can be adjusted to generate concentric standing waves in the vessel. However, any convenient type or number of ultrasonic transducers may be used, such as using ultrasound horns, and employed in ways which are routine to those skilled in the art.

We have also found that the technique works at a wide range of ultrasound frequencies. It is however preferred to introduce the ultrasound into the composition when the system is in resonance, since this results in the most effective and efficient transfer of ultrasound to the composition.

The method of the invention also works at a wide range of intensities (i.e. typically from 0.1 to 40-50 dB or higher) using typical (eg wax) based compositions in which the wax structures a carrier fluid such as for example a volatile silicone. Cavitation may typically be induced in such a composition at an intensity of around 30-40 dB, the threshold intensity varying with the nature of the relevant constituents of the formulation. However, the method works satisfactorily and the resultant products are also satisfactory below the threshold intensity. This is a benefit for producing the instant products, ie cosmetic sticks based upon a structurant in a carrier fluid, in that it enables a wide range of intensities to be employed.

In terms of the ultrasound conditions employed, typically a frequency of 11 kHz may be used. Conveniently, an intensity of ultrasound of 20 to 30 dB may also be used.

Exposure times of the composition may vary and will depend on the nature of the rig employed and whether the user wishes to harden or soften the stick. Exposure times to harden the stick may typically be in the region of 5 seconds to 5 minutes, preferably 10 seconds to 3 minutes, and will in practice be any duration necessary to produce a harder stick, or an increase in solidification temperature. Hardening processes according to the invention will be characterised by an increase in the hardness or solidification temperature of the cosmetic stick produced, compared to the hardness or solidification temperature of the corresponding stick when manufactured without exposing the composition to ultrasound.

Exposure times to soften the stick may typically be in the region of 5 minutes to 45 minutes, preferably 5 minutes to 30 minutes, and will in practice be any duration necessary to produce a stick of the same or reduced hardness after an initial increase in hardness, or an increase in solidification temperature. Softening processes according to the invention will typically be characterised by a decrease in the hardness or solidification temperature of the cosmetic stick produced, compared to the hardness of the corresponding stick when manufactured without exposing the composition to ultrasound.

When the sticks are exposed to ultrasound for long enough to form soft solids, the process can be conducted, if desired, together with a shearing process, such as high shear mixing.

Where the cosmetic stick produced according to the method of the invention is a suspension antiperspirant stick, the stick may comprise conventional ingredients.

A typical ingredient may be a carrier liquid which in conjunction with the organic crystalline structurant may provide the vehicle of the stick. A carrier liquid may typically be water immiscible, and may comprise one or a mixture of materials which are relatively hydrophobic so as to be immiscible in water. Some hydrophilic liquid may be included in the vehicle, provided the overall vehicle liquid mixture is sufficiently immiscible with water such that any antiperspirant active remains undissolved. It will often be desired that this vehicle is liquid (in the absence of structurant) at temperatures of 15° C. and above. It may have some volatility, but its vapour pressure will generally be less than 4 kPa (30 mm Hg) at 25° C. so that the material can be referred to as an oil or mixture of oils. Typically, it may be that at least about 80% by weight of the hydrophobic vehicle liquid should consist of materials with a vapour pressure not over this value of 4 kPa at 25° C.

It is preferred that the hydrophobic vehicle material includes a volatile liquid silicone, i.e. liquid polyorganosiloxane. To be classed as “volatile” such material should have a measurable vapour pressure at 20 or 25° C. Typically the vapour pressure of a volatile silicone lies in a range from 1 or 10 Pa up to 2 kPa at 25° C.

It is desirable to include a volatile silicone because it gives a “drier” feel to the applied film after the composition is applied to skin.

Volatile polyorganosiloxanes can be linear or cyclic or mixtures thereof. Preferred cyclic siloxanes include polydimethsiloxanes and particularly those containing from 3 to 9 silicon atoms and preferably not more than 7 silicon atoms and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 10⁻⁵ m²/sec (10 centistokes), and particularly above 10⁻⁷ m²/sec (0.1 centistokes), the linear siloxanes normally exhibiting a viscosity of below 5×10⁻⁶ m²/sec (5 centistokes). The volatile silicones can also comprise branched linear or cyclic siloxanes such as the aforementioned linear or cyclic siloxanes substituted by one or more pendant -0-Si(CH₃)₃ groups. Examples of commercially available silicone oils include oils having grade designations 344, 345, 244, 245 and 246 from Dow Corning Corporation; Silicone 7207 and Silicone 7158 from Union Carbide Corporation; and SF1202 from General Electric. The hydrophobic vehicle liquid employed in compositions herein can alternatively or additionally comprise non-volatile silicone oils, which include polyalkyl siloxanes, polyalkylaryl siloxanes and polyethersiloxane copolymers. These can suitably be selected from dimethicone and dimethicone copolyols. Commercially available non-volatile silicone oils include Dow Corning 556 and Dow Corning 200 series.

The water-immiscible liquid vehicle may contain from 0 to 100% by weight of one or more liquid silicones. Preferably, there is sufficient liquid silicone to provide at least 10%, better at least 15%, by weight of the whole composition. If silicone oil is used, volatile silicone preferably lies in a range from 20% possibly from 30 or 40% up to 100% of the weight of the water-immiscible carrier liquid. In many instances, when a non-volatile silicone oil is present, its weight ratio to volatile silicone oil is chosen in the range of from 1:3 to 1:40.

Silicon-free hydrophobic liquids can be used instead of, or more preferably in addition to liquid silicones. Silicon-free hydrophobic organic liquids which can be incorporated include liquid aliphatic hydrocarbons such as mineral oils or hydrogenated polyisobutene, often selected to exhibit a low viscosity. Further examples of liquid hydrocarbons are polydecene and paraffins and isoparaffins of at least 10 carbon atoms.

Other hydrophobic vehicle liquids are liquid aliphatic or aromatic esters. Suitable aliphatic esters contain at least one long chain alkyl group, such as esters derived from C₁ to C₂₀ alkanols esterified with a C₈ to C₂₂ alkanoic acid or C₆ to C₁₀ alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate.

Suitable liquid aromatic esters, preferably having a melting point of below 20° C., include fatty alkyl benzoates. Examples of such esters include suitable C₈ to C₁₈ alkyl benzoates or mixtures thereof.

Further instances of suitable hydrophobic vehicle liquids comprise liquid aliphatic ethers derived from at least one fatty alcohol, such as myristyl ether derivatives e.g. PPG-3 myristyl ether or lower alkyl ethers of polyglycols such as PPG-14 butyl ether.

Where the cosmetic stick is a suspension antiperspirant stick, the vehicle may also contain relatively minor amounts of hydrophilic liquids, such as water.

However, a further suitable class of water soluble or water-miscible liquids comprises short chain monohydric alcohols, e.g. C₁ to C₄ and especially ethanol or isopropanol, which can impart a deodorising capability to the formulation. A further class of hydrophilic liquids comprises diols or polyols preferably having a melting point of below 40° C., or which are water miscible. Examples of water-soluble or water-miscible liquids with at least one free hydroxy group include ethylene glycol, 1,2-propylene glycol, 1,3-butylene glycol, hexylene glycol, diethylene glycol, dipropylene glycol, 2-ethoxyethanol, diethylene glycol monomethylether, triethyleneglycol monomethylether and sorbitol. Especially preferred are propylene glycol and glycerol.

Where hydrophilic liquids are incorporated in suspension antiperspirant sticks, their levels are generally maintained sufficiently low so as not to cause substantial dissolution of the suspended antiperspirant active.

Where the topical composition is an antiperspirant stick, it will contain an antiperspirant active.

Antiperspirant actives are preferably incorporated in an amount of from 0.5-60%, particularly from 5 to 30% or 40% and especially from 5 or 10% to 30 or 35% of the weight of the whole composition.

Antiperspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula Al₂(OH)ₓQ_{y}.wH₂O in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever NV et al), the contents of which specification is incorporated herein by reference. Some activated salts do not retain their enhanced activity in the presence of water but are useful in substantially anhydrous formulations, i.e. formulations which do not contain a distinct aqueous phase.

Zirconium actives can usually be represented by the empirical general formula: ZrO(OH)_{2n-nz}B_{z}.wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by w H₂O. Preferable is that B represents chloride and the variable z lies in the range from 1.5 to 1.87. In practice, such zirconium salts are usually not employed by themselves, but as a component of a combined aluminium and zirconium-based antiperspirant.

The above aluminium and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-é-phenylalanine, d1-valine, dl-methionine and é-alanine, and preferably glycine which has the formula CH₃CH(NH₂)CO₂H.

It is highly desirable to employ complexes of a combination of aluminium halohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium,zirconium and chloride with an Al/Zr ratio in a range from 2 to 10, especially 2 to 6, an Al/Cl ratio from 2.1 to 0.9 and a variable amount of glycine. Actives of this preferred type are available from Westwood, Summit and Reheis. When further activated, such glycine complexed antiperspirant actives are often referred to as AZAG.

Other actives which may be utilised include astringent titanium salts, for example those described in GB 2299506A.

The proportion of solid antiperspirant salt in the composition normally includes the weight of any water of hydration and any complexing agent that may also be present in the solid active. However, when the active salt is in solution, its weight excludes any water present.

The antiperspirant active will often provide from 3 to 60% by weight of the total composition, particularly from 5% or 10% up to 30% by weight of the total composition. Optional ingredients in topical antiperspirant compositions of this invention can include disinfectants, for example at a concentration of up to about 10% w/w. Suitable deodorant actives can comprise deodorant effective concentrations of antiperspirant metal salts, deoperfumes, and/or microbicides, including particularly bactericides, such as chlorinated aromatics, including biguanide derivatives, of which materials known as Igasan DP300™, Triclosan, TriclobanTM, and Chlorhexidine warrant specific mention. A yet another class comprises biguanide salts such as those available under the trade mark Cosmosil™.

Other optional ingredients include wash-off agents, often present in an amount of up to 10% w/w to assist in the removal of the formulation from skin or clothing. Such wash-off agents are typically nonionic surfactants such as esters or ethers containing a C₈ to C₂₂ alkyl moiety and a hydrophilic moiety which can comprise a polyoxyalkylene group (POE or POP) and/or a polyol.

The compositions herein can incorporate one or more cosmetic adjuncts conventionally contemplatable for antiperspirant solids or soft solids. Such cosmetic adjuncts can include skin feel improvers, such as talc or finely divided polyethylene, for example in an amount of up to about 10%; skin benefit agents such as allantoin or lipids, for example in an amount of up to 5%; colours; skin cooling agents other than the already mentioned alcohols, such a menthol and menthol derivatives, often in an amount of up to 2%, all of these percentages being by weight of the composition. A commonly employed adjunct is a perfume, which is normally present at a concentration of from 0 to 4% and in many formulations from 0.25 to 2% by weight of the composition.

Where hydrophilic liquids are incorporated in suspension antiperspirant sticks, the levels are generally maintained sufficiently low so as to not cause substantial dissolution of the suspended antiperspirant active.

A generalised conventional manufacturing method for suspension antiperspirant sticks, the majority of the components are first melted, and the antiperspirant active and then the other additives are added as appropriate. The composition is then dosed whilst still mobile. Finally the composition is cooled in a cooling tunnel until the composition is solidified.

The cooling and solidification time increases the overall batch time and represents an expensive bottleneck in the batch process. Therefore it is a very important benefit of the invention to be able to cool the molten fluid composition rapidly, so that solidification times can be reduced.

It is also necessary to cool the liquid rapidly to ensure that the active ingredient is evenly distributed throughout the solid. If the cooling is too slow the active ingredient settles towards the base of the stick, and as a result is unevenly distributed throughout the solid stick that is formed. The efficiency of the product may be reduced if the active ingredient is unevenly distributed throughout the antiperspirant stick.

The invention will now be further by way of example only, with reference to the accompanying drawings, in which:

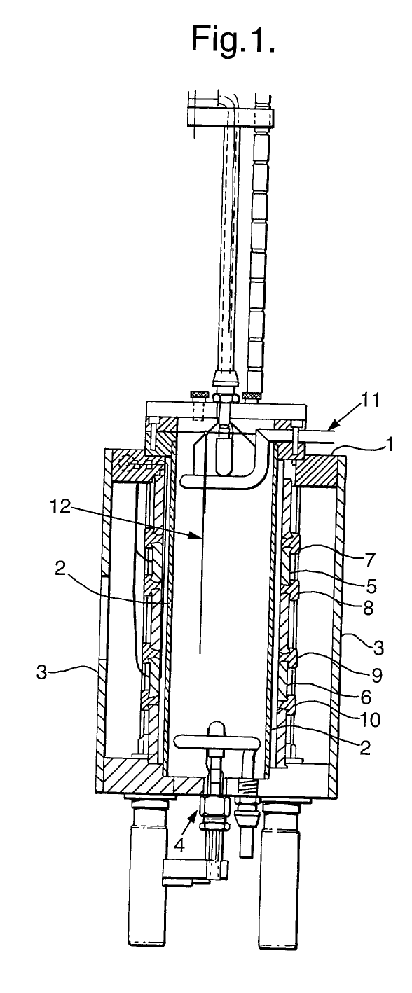

FIG. 1 represents a suitable ultrasound inducing rig for use according to the invention; and

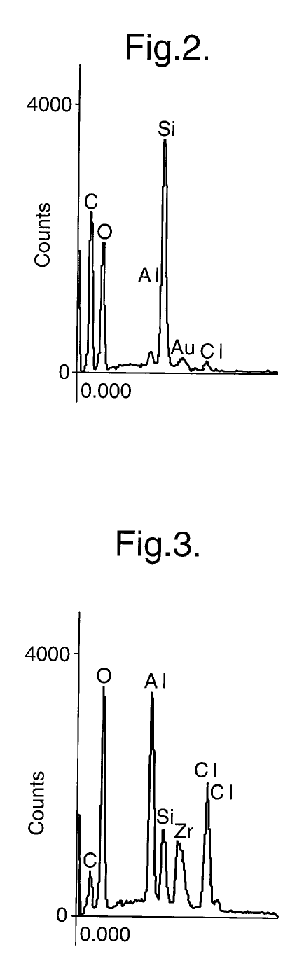

FIGS. 2 and 3 graphs of the elemental compositions of discreet particles in stick cross sections made by conventional melt cast techniques (FIG. 2) and according to the invention (FIG. 3).

### EXAMPLE 1

The solidification of antiperspirant sticks at a higher temperature was induced using ultrasound.

A suitable rig for preparing compositions according to the invention comprises is shown in FIG. 1 and comprises a vessel 1 comprising an inner Perspex™ jacket 2 and an outer Perspex jacket 3. The vessel 1 is generally cylindrical and closed at both ends. Projecting into the body of the vessel 1 through one of the ends is a thermocouple arrangement 4 which also has combined with it a hydrophone arrangement to measure the intensity of the ultrasound within the vessel.

Projecting into the body of the vessel 1 at the other end are cooling/heating coils 11, fixed thermocouples 12 and also a blade stirrer.

Located circumferentially around the periphery of the inner Perspex™ jacket 2 are two circular transducers 5, 6 which are held in place by alignment rings 7, 8, 9, 10.

Ultrasound introduced via transducers 5, 6 may be generated and controlled by standard generating and amplifying equipment which is readily commercially available, which adjusts the frequency and intensity of the ultrasound as appropriate.

The frequency of the ultrasound in the apparatus is adjusted and monitored via the hydrophone such that a resonant frequency is detected and maintained. For the apparatus shown in FIG. 1, the resonant frequencies were found to be in the region 10-11 kHz and 66-70 kHz. The lower frequency was preferred in this embodiment, since it was found that the higher frequency band had an acoustic amplitude which decreased in the operating temperature range.

In use, molten antiperspirant stick composition is introduced into the rig, the ultrasound applied, and the ultrasonicated composition is then transferred into barrels.

A typical formulation of suspension antiperspirant stick suitable for processing in the above apparatus is as follows:

| | | % w/w |
|---|---|---|
| DC345 | Volatile Silicone | 53 |
| Castorwax (MP80) | | 4 |
| Lanette C18 Deo | Stearyl alcohol | 14 |
| Estol EO4DS | PEG-8 distearate | 1 |
| AZAG powder | Antiperspirant | 24 |
| | active | |
| Talc | | 3 |
| Perfume | | 1 |

The fatty alcohol, castor oil, silicone oil and other miscellaneous additives were melted at a temperature range of 80° C. or more. The mixture was then allowed to cool to 70° C., and the active ingredient and perfume were added. The mixture was then exposed to ultrasound whilst being pumped at a temperature between 67 and 70° C., and the product solidified more rapidly on being dosed into barrels. This exposure of the material to ultrasound represents a modification of a routine melt casting technique.

The frequency of the ultrasound was between 10 and 66 kHz and the treatment time was between 5 and 60 seconds.

The solids formed by exposure of the liquid to ultrasound had relatively uniform structures in which the active ingredient was evenly distributed. The uniform structure improves the mechanical and sensory properties of the antiperspirant stick. The even distribution of the active ingredient improves the efficiency of the product.

It was also surprisingly found that after exposure to ultrasound, there was no deterioration of the most fragile chemical species (ie the perfume) in the composition, even when the composition, even when the composition was subject to ultrasound intensities above cavitation (ie up to about 40 dB).

When processed by routine melt casting techniques, the composition has a solidification temperature of 52° C., and a hardness of 0.28 N.mm⁻², ±0.03 N.mm⁻². However, exposing the composition to ultrasound during the mixing and cooling stage it was possible to produce sticks with a hardness as high as 0.42 N.mm⁻². It was also possible to alter the solidification temperature of the composition to be as high as 73° C., by varying the process parameters.

### EXAMPLE 2

In Example 2, a stick having reduced hardness was obtained using the general process and ultrasonic apparatus described for Example 1 in respect of the same formulation, but with the frequency and treatment time shown below.

The frequency of the ultrasound was between 10 and 66 kHz and the treatment time was between 5 and 10 minutes.

The solids formed by exposure of the liquid to ultrasound had relatively uniform structures in which the active ingredient was evenly distributed. The uniform structure improves the mechanical and sensory properties of the antiperspirant stick. The even distribution of the active ingredient improves the efficiency of the product.

It was also surprisingly found that after exposure to ultrasound, there was no deterioration of the most fragile chemical species (ie the perfume) in the composition, even when the composition, even when the composition was subject to ultrasound intensities above cavitation (ie up to about 40 dB).

When processed by routine melt casting techniques, the composition has a solidification temperature of 52> C., and a hardness of 0.28 N.mm⁻² ±0.03 N.mm⁻². However, exposing the composition to ultrasound during the mixing and cooling stage it was possible to produce sticks with a hardness as low as 0.18 N.mm⁻², the composition initially having become harder before peaking in its hardness and eventually becoming softer than the comparable melt cast stick. It was also possible to alter the solidification temperature of the composition to be as high as 73° C., by varying the process parameters.

Measurement of Properties in Examples 1 and 2

Stick hardnesses were measured by advancing the test stick out of its barrel a short distance to reveal the curved head. A sharp blade was then used to cut a flat surface on the top of the stick. The stick was then wound back inside the barrel to prevent slack.

Hardness was assessed using an Instron 5566 tester. The barrel was placed on the bottom fixed platen of the instrument with the sectioned face pointing upwards. The top moving platen was fixed with a 2.5N load cell with an attachment bearing a 4.76 mm sapphire sphere.

The sapphire sphere was driven into the sectioned face at a velocity of 50 μm/s until a load of 2N was attained. At this point the top platen section was reversed, and the sphere drawn away from the stick at the same velocity. The loading and displacement data was logged. The procedure was repeated three times on different parts of the stick face, and the results averaged.

### EXAMPLE 3

Sticks of the composition described in Example 1 produced according to the invention were compared to similar composition sticks made by melt casting techniques by subjecting them to freeze fracture, and then examining the fractured surface of the stick by cryo-scanning electron microscopy.

According to the method, oval cross section stick compositions were formed, and then laterally fractured at −70> C. The fractured surfaces of the sticks were then examined by cryo-scanning microscopy, focusing on small domains on the fracture surface which appear to be discreet particles. A large number of such domains are surveyed such that the analysis of each stick fracture surface can be considered to be statistically significant.

For each domain examined, the domain is analysed by semi quantitative electron differential scanning techniques. It was found that for discreet domains which appear to be particles in sticks produced according to the invention, relatively prominent peaks were observed for aluminium and zirconium relative to the carbon peak for the sticks made using ultrasound, compared to those made using conventional melt cast techniques. This indicates that on any given freeze fracture surface, the aluminium/zirconium antiperspirant active particles are much closer to the surface than in those sticks made by melt casting. This in turn indicates a much lower degree of enrobing of the aluminium/zirconium active particles in sticks made using ultrasound, compared to those made conventionally.

Electron differential scanning graphs for sticks made conventionally and using ultrasound are shown as FIGS. 2 and 3 respectively.

We further observed that sticks made according to melt cast techniques had a relatively high concentration of stearyl alcohol in the chill zone, and their lowest concentration of stearyl alcohol in the dendritic zone.

In contrast sticks according to the invention had a uniform concentration of stearyl alcohol across their entire cross section.

We claim:

1. A method of inducing solidification in a fluid composition comprising crystalline organic structurant, comprising exposing the fluid composition to ultrasound.

2. A method according to claim 1, wherein the method is for manufacturing cosmetic sticks and includes the steps of providing a molten composition of the cosmetic stick containing the organic structurant which is crystalline when the composition is solid, the molten formulation optionally containing solid particles, cooling the molten composition to a temperature at which it is solid, and subjecting the molten composition to ultrasound prior to solidification of the composition.

3. A method according to claim 2, wherein the fluid is a liquid, a solution, a suspension or an emulsion.

4. A method according to claim 2 including the further step of forming the fluid composition into a solidified topical product which is a stick, soft solid or cream.

5. A method according to claim 2 wherein the hardness of the resultant stick, soft solid or cream is higher after the exposure to ultrasound than the same composition of stick made without exposure to ultrasound.

6. A method according to claim 2 wherein the hardness of the resultant stick, soft solid or cream is lower after the exposure to ultrasound than the same composition of stick made without exposure to ultrasound.

7. A method according to claim 2, wherein the crystalline organic structurant is based on a compound containing a high proportion of hydrocarbon chains.

8. A method according to claim 7, wherein the crystalline organic structurant is a wax.

9. A method according to claim 8, wherein the wax does not form fibres on solidification.

10. A method according to claim 8 wherein the wax has a needle, plate or spherulite crystalline form.

11. A method according to claim 8, wherein the wax is selected from paraffin waxes, Synchrowax™ HRC, carnabau, beeswax, modified beeswaxes, microcrystalline waxes, polyethylene waxes and fatty ester derivatives of polyols such as glycerol monostearate and related compounds, spermacetti, candelilla and silicone waxes, fatty alcohols, fatty acids, esters containing a fatty acid or alcohol residue, fatty being linear and waxy hydrocarbons, and mixture thereof.

12. A method according to claim 11, wherein the wax is castor wax.

13. A method according to claim 2, wherein the crystalline organic structurant is at least partially liquid in the fluid composition.

14. A method according to claim 2, wherein the solidification temperature of the fluid composition is raised by 1° C. or more.

15. A method according to claim 14, wherein the solidification temperature of the fluid composition is raised by 3° C. or more.

16. A cosmetic stick according to claim 2 in which the water-immiscible carrier comprises a volatile silicone.

17. A cosmetic stick for topical application to the human skin made according to the method of any of claims 1 to 15.

18. A cosmetic stick according to claim 16 wherein the stick is an antiperspirant or deodorant stick.

19. Use of ultrasound to induce solidification in a fluid composition comprising an organic crystalline structurant, a cosmetic active and a water-immiscible carrier.

20. Use according to claim 17 for the purpose of manufacturing a cosmetic stick.

21. Use according to claim 18 wherein the cosmetic stick is an antiperspirant or deodorant stick.

22. A cosmetic composition comprising a cosmetic active suspended in a carrier fluid containing a crystalline organic structurant characterised in that the structurant is present in the form of domains that are deficient in the cosmetic active.

23. A cosmetic stick composition for topical application to the human skin comprising a suspended active which comprises a non-carbon element and a hydrophobic carbon containing matrix, characterised in that when the stick is analysed by cryo scanning microscopy techniques, the active generates a characteristic peak from both the non-carbon active element and the carbon containing hydrophobic matrix, and the ratio of the size of the non-carbon active peak to carbon peak in the scanning micrograph is higher than when the same composition of stick is made by melt casting techniques.

24. A cosmetic stick according to claim 22 or 23, wherein the cosmetic stick is an antiperspirant stick and the active is an antiperspirant active.

25. A cosmetic stick according to any of claims 22 or 23, wherein the non-carbon active element is a transition metal or a group III metal.

26. A cosmetic stick according to claim 25, wherein the active comprises aluminium, zirconium or titanium.

27. A cosmetic stick according to claim 22 or 23, manufactured using ultrasound.

28. A cosmetic stick according to claim 27, wherein the ultrasound is applied to a fluid composition of the stick composition whilst it is cooled.

29. A cosmetic stick according to claim 27, wherein the ultrasound is applied to the stick composition until it attains soft solid form, optionally in conjunction with shearing.
